SEQUENCE LISTING (17 sequences)

SEQ ID NO: 1 - DNA, 20 bp, Mycobacterium tuberculosis
cttgcacgagggtcagacca

SEQ ID NO: 2 - DNA, 20 bp, Mycobacterium tuberculosis
atctcgtcgctaaccacgcc

SEQ ID NO: 3 - DNA, 20 bp, Mycobacterium tuberculosis
aacgacgtcgaaacagcggc

SEQ ID NO: 4 - DNA, 20 bp, Mycobacterium tuberculosis
gcgaactcgtcggccaattc

SEQ ID NO: 5 - DNA, 23 bp, Mycobacterium tuberculosis
tgcccagaaagggatccgtcatg

SEQ ID NO: 6 - DNA, 20 bp, Mycobacterium tuberculosis
atgaggaatgcgtccgcgga

SEQ ID NO: 7 - DNA, 19 bp, Mycobacterium tuberculosis
gccgcgatcaaggagttct

SEQ ID NO: 8 - DNA, 17 bp, Mycobacterium tuberculosis
acgtcgcggacctccag

SEQ ID NO: 9 - DNA, 19 bp, Mycobacterium tuberculosis
ccaccatacggatagggga

SEQ ID NO: 10 - DNA, 15 bp, Mycobacterium tuberculosis
tggaccgccagggct

SEQ ID NO: 11 - DNA, 19 bp, Mycobacterium tuberculosis
aattcatggtccagaacaa

SEQ ID NO: 12 - DNA, 14 bp, Mycobacterium tuberculosis
gttgaccctcaagc

SEQ ID NO: 13 - DNA, 18 bp, Mycobacterium tuberculosis
tcgtatggcaccggaacc

SEQ ID NO: 14 - DNA, 18 bp, Mycobacterium tuberculosis
cagctcccactcgtagcc

SEQ ID NO: 15 - DNA, 16 bp, Mycobacterium tuberculosis
atcaccaccggcatcg

SEQ ID NO: 16 - DNA, 20 bp, Mycobacterium tuberculosis
cgttacgctcgtggacatac

SEQ ID NO: 17 - DNA, 19 bp, Mycobacterium tuberculosis
gtttcctccggtaaccagg

(19) United States
(12) Patent Application Publication
(10) Pub. No.: US 2013/0095489 A1
(43) Pub. Date: Apr. 18, 2013

(54) PROCESS FOR DETECTION OF MULTIDRUG RESISTANT TUBERCULOSIS USING REAL-TIME PCR AND HIGH RESOLUTION MELT ANALYSIS

(71) Applicants: James Posey, Atlanta, GA (US); Jonas Winchell, Lilburn, GA (US); Kelley Cowart, Athens, GA (US); Melissa Ramirez, Fort Collins, CO (US)

(72) Inventors: James Posey, Atlanta, GA (US); Jonas Winchell, Lilburn, GA (US); Kelley Cowart, Athens, GA (US); Melissa Ramirez, Fort Collins, CO (US)

(73) Assignee: CENTERS FOR DISEASE CONTROL AND PREVENTION, Atlanta, GA (US)

(21) Appl. No.: 13/695,935

(22) PCT Filed: May 4, 2011
(86) PCT No.: PCT/US2011/035217

§ 371 Date: Nov. 2, 2012

Related U.S. Application Data
(60) Provisional application No. 61/331,189, filed on May 4, 2010.

(51) Int. Cl. C12Q 1/68

(52) U.S. Cl. CPC C12Q 1/689
USPC 435/6.11; 435/6.12

(57) ABSTRACT

Compositions and process are provided for the rapid and specific detection of drug resistant forms of Mycobacterium tuberculosis based on real time PCR and high resolution melt analysis. The compositions and processes are useful for the detection of mutations within the Rifampicin Resistance Determinant Region (RRDR) of rpoB for the detection of rifampicin (RIF) and within specific regions of katG and the inhA promoter for the detection of isoniazid (INH) resistance. The invention also is capable of rapidly discriminating Mycobacterium tuberculosis complex (MTBC) strains from Nontuberculous Mycobacteria (NTM) strains.

### REFERENCE TO RELATED APPLICATION

This application claims priority from U.S. Provisional Patent Application Ser. No. 61/331,189, filed May 4, 2010, the entire content of which is incorporated herein by reference.

### GOVERNMENT INTEREST

The invention described herein may be manufactured, used, and licensed by or for the United States Government.

### FIELD OF THE INVENTION

The invention relates generally to a process of detecting drug resistant Mycobacterium tuberculosis based on real time PCR and high resolution melt analysis; and in particular, to the detection of mutations within the Rifampicin Resistance Determinant Region (RRDR) of rpoB and specific regions of katG and the inhA promoter for the detection of rifampicin (RIF) and isoniazid (INH) resistance, respectively. The invention also is capable of discriminating Mycobacterium tuberculosis complex (MTBC) strains from Nontuberculous Mycobacteria (NTM) strains.

### BACKGROUND OF THE INVENTION

The World Health Organization (WHO) estimates that approximately one-third of the world's population is infected with Mycobacterium tuberculosis (Mtb), with an estimated 9.27 million new cases reported in 2007 (20). In that year alone, an estimated 1.77 million people died from this treatable disease. Despite this significant burden, only a limited number of tests have been developed and implemented for the rapid diagnosis of tuberculosis (TB). Further, since the majority of TB disease burden occurs in under-developed and resource-limited settings, the need for a cost-efficient method is paramount.

The emergence of drug resistant strains of Mtb is one of the most critical issues facing TB researchers and clinicians today. Multidrug resistant TB (MDR-TB) is defined as being resistant to the two best first line drugs used to treat TB: rifampicin (RIF) and isoniazid (INH). Extensively drug resistant TB (XDR-TB) is defined as having additional resistance to a fluoroquinolone (ciprofloxacin, moxifloxicin, etc.) and an injectable (kanamycin, capreomycin, or amakacin), the two best classes of second line drugs. The WHO estimates that 5% of new TB cases are MDR, with approximately 10% of those actually being XDR (20). Compounding this problem is the fact that no new drugs have been developed and approved for the treatment of TB in the past thirty years (16). The limited number of antibiotics available to treat TB necessitates rapid diagnosis not only to reduce the spread of drug resistant strains, but also to monitor and limit the emergence of newly resistant strains.

While RIF and INH are very effective in the treatment of susceptible strains of Mtb, drug resistance can emerge quickly, in part, due to non-adherence to the multidrug regimen or non-continuous treatment. The molecular basis of resistance to these drugs is well documented. The target of RIF is the beta-subunit of bacterial DNA-dependent RNA polymerase, which is encoded by the rpoB gene. At the genetic level, the majority of RIF resistance is due to the accumulation of mutations within an 81 base pair (bp) region of rpoB, termed Rifampicin Resistance Determinant Region (RRDR). Mutations within this region account for up to 98% of the RIF resistance observed (15). The strong correlation between genotypic changes in this region resulting in phenotypic resistance makes the RRDR an optimal target for the design of rapid molecular diagnostics.

There are two described mechanisms that account for the majority of INH resistance. The most common mechanism involves mutations within the katG gene that encodes a catalase peroxidase whose activity is required for the activation of INH (9). Nucleotide changes resulting in amino acid substitutions at codon 315 of katG account for up to 50% of the clinical resistance to INH (15). Another less common mutation occurs in the promoter region of the inhA gene that encodes the enoyl-ACP reductase that is required for mycolic acid biosynthesis (18). Mutations at this locus account for up to 34% of clinical INH resistance observed and are typically found in combination with additional mutations in katG (15).

The vast majority of mutations that occur within rpoB, katG, and the inhA promoter regions are due to accumulation of single nucleotide polymorphisms (SNPs), of which there are four classes (8). Class I SNPs, also called transitions, are changes in which a purine is exchanged for a purine (A/G>G/A), or a pyrimidine is exchanged for a pyrimidine (C/T>T/C) (8). Classes II, III, and IV SNP changes are collectively referred to as transversions and all involve the change of a purine to a pyrimidine, or vice versa (17). Class II changes result in A/C>C/A or T/G>G/T transversions, Class III changes result in C/G>G/C transversions, and class IV changes result in A/T>T/A changes (8). These genetic mutations often result in phenotypic changes, such as RIF and INH resistance observed in Mtb, and are excellent targets for rapid molecular diagnostics.

A significant obstacle in controlling TB is the amount of time required to reach a diagnosis. Due to the slow growth rate of Mtb, the initial diagnosis can take up to six weeks, with up to an additional 12 weeks to obtain drug susceptibility profiles for clinical isolates depending on the techniques available to the laboratory. These labor-intensive methods can cause significant delays in identifying MDR or XDR cases, adjusting treatment regimens, and initiating epidemiological investigations. Recently, attention has shifted towards the development of dependable, molecular-based assays that can rapidly detect drug resistance. The development of new methodologies could potentially reduce the time required to diagnose drug resistance so that effective treatment regimens can be established. Direct sequencing of genes known to have a role in antibiotic resistance is one method that is currently used. However, while reliable, it is costly and may not be readily available. Another rapid method, the GenoType MTBDRplus assay (Hain Lifescience GmbH, Nehren, Germany), has made substantial contributions to the area of rapid diagnostics, but still requires approximately eight hours to complete the assay and additional training to ensure that results are interpreted correctly (7).

Thus, there exists a need for compositions, processes and technology to detect RIF resistant, INH resistant, and importantly, MDR strains of Mtb. There further exists a need for an assay that is capable of distinguishing Mycobacterium tuberculosis complex bacteria (MTBC) from Nontuberculous Mycobacteria (NTM) strains.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

A process for detecting drug resistance in a Mycobacterium tuberculosis strain is provided including exposing a DNA sample to a primer pair that includes a forward primer and a reverse primer under conditions conducive to a polymerase chain reaction to yield an amplicon, wherein the primer pair is specific to a region of rpoB, katG or inhA promoter of the Mycobacterium tuberculosis strain; and detecting the amplicon indicative of the Mycobacterium tuberculosis strain. In some embodiments, the primer pair is: SEQ ID NO: 7 and SEQ ID NO: 8; SEQ ID NO: 13 and SEQ ID NO: 14, or SEQ ID NO: 16 and SEQ ID NO: 17, or combinations of primer pairs. Some embodiments further include exposing the sample to an IS6110 insertion element forward primer and an IS6110 insertion element reverse primer under the conditions conducive to polymerase chain reaction to yield an amplicon and to determine a strain as a MTBC or NTM strain of mycobacteria. An IS6110 insertion element forward primer optionally has the sequence of SEQ ID NO: 9. An IS6110 insertion element reverse primer optionally has the sequence of SEQ ID NO: 10.

An inventive process optionally includes performing high resolution melt analysis on a double-stranded product comprising the amplicon. In some embodiments, high resolution melt analysis is performed from 60 degrees Celsius to 90 degrees Celsius. This range or a subrange thereof is sufficient to detect and distinguish a strain as a MTBC or NTM strain of mycobacteria. In some embodiments, a high resolution melt analysis is performed between 80 degrees Celsius and 89 degrees Celsius or any subrange thereof. The high resolution melt analysis is optionally performed at a rate of 0.02 degrees Celsius per step.

A process optionally includes exposing the sample or the amplicon to one or more probes targeting one or more specific loci in at least one of rpoB, katG or inhA promoter. The probe optionally contains one or more locked nucleic acids. In some embodiments, a katG probe has the sequence of SEQ ID NO: 15. In some embodiments, the rpoB probe has the sequence of SEQ ID NO: 11 or SEQ ID NO: 12. A single polymerase chain reaction optionally includes primers for rpoB, katG or inhA promoter regions as well as probes targeting one or more specific loci in at least one of rpoB, katG or inhA promoter, or combinations thereof. An inventive process optionally allows amplification of at least a portion of rpoB, katG and the inhA promoter in a single polymerase chain reaction.

A DNA sample, an amplicon, or both are optionally subjected to a DNA sequencing reaction. A DNA sequencing reaction optionally uses a primer pair selected from the sequences of SEQ ID NO: 1 and SEQ ID NO: 2; SEQ ID NO: 3 and SEQ ID NO: 4, or SEQ ID NO: 5 and SEQ ID NO: 6; or combinations of the pairs.

A DNA sample is optionally obtained by selective amplification of a region of a Mycobacterium tuberculosis strain genome. Selective amplification is optionally by a polymerase chain reaction using primer pairs that specifically amplify a region of the Mycobacterium tuberculosis strain genome. A region of a Mycobacterium tuberculosis strain genome is optionally the or a region of rpoB, katG or inhA promoter. Primer pairs optionally include at least one of any of SEQ ID NO: 1-6.

Also provided is a process of distinguishing a Mycobacterium tuberculosis complex bacteria from a nontuberculosis Mycobacteria including exposing a DNA sample from the bacteria to a first primer pair specific to a rpoB region of the bacteria under conditions conducive to a polymerase chain reaction to yield a first amplicon; exposing the DNA sample to a second primer pair specific to an IS6110 insertion element of Mycobacterium tuberculosis under conditions conducive to a polymerase chain reaction to yield a second amplicon; and detecting the presence or absence of a Mycobacterium tuberculosis complex bacteria amplicon. Optionally included is performing high resolution melt analysis on the first amplicon and the second amplicon. In some embodiments, a second primer pair has a forward primer with the sequence of SEQ ID NO: 9 and a reverse primer with the sequence of SEQ ID NO: 10. In some embodiments, a first primer pair has a forward primer with the sequence of SEQ ID NO: 7 and a reverse primer with the sequence of SEQ ID NO: 8. Detection of two separate melting peaks in an high resolution melt analysis is indicative of a Mycobacterium tuberculosis strain.

Kits for detecting a Mycobacterium tuberculosis strain are also provided. A kit includes a forward primer—reverse primer pair that will selectively amplify at least a region of rpoB, katG or inhA promoter, or combinations thereof. In some embodiments, a forward and reverse primer pair includes at least one of SEQ ID NO: 7, SEQ ID NO: 8, SEQ ID NO: 13, SEQ ID NO: 14, SEQ ID NO: 16, SEQ ID NO: 17 or combinations thereof. A kit optionally includes a detectable probe specific for specific SNPs in a Mycobacterium tuberculosis strain or generic to more than one Mycobacterium tuberculosis strain. A probe optionally has the sequence of SEQ ID NO: 11, SEQ ID NO: 12, or SEQ ID NO: 15. A kit optionally also includes one or more of a primer having a sequence of SEQ ID NO: 1, SEQ ID NO: 2, SEQ ID NO: 3, SEQ ID NO: 4, SEQ ID NO: 5, SEQ ID NO: 6.

Also provided is a process for distinguishing a Mycobacterium tuberculosis complex bacteria from a nontuberculosis Mycobacteria including exposing a DNA sample to three or more primer pairs where at least one primer pair is specific for at least one of rpoB, katG or inhA promoter. The presence of a single amplicon representing rpoB, katG and inhA promoter indicates the presence of Mycobacterium tuberculosis complex bacteria and excludes a nontuberculosis Mycobacteria in the sample. High resolution melt analysis is optionally performed to distinguish the single amplicons or identify more than one amplicon for a single primer pair. A first primer pair is optionally SEQ ID NO: 7 and SEQ ID NO: 8. A first primer pair is optionally SEQ ID NO: 13 and SEQ ID NO: 14. A first primer pair is optionally SEQ ID NO: 16 and SEQ ID NO: 17. The process optionally includes exposing the sample to an IS6110 insertion element forward primer and an IS6110 insertion element reverse primer under the conditions conducive to polymerase chain reaction to yield a fourth amplicon. An IS6110 insertion element forward primer optionally has the sequence of SEQ ID NO: 9. An IS6110 insertion element reverse primer optionally has the sequence of SEQ ID NO: 10.

Inventive nucleotide sequences are optionally provided. An inventive nucleotide sequence has a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

### BRIEF DESCRIPTION OF THE DRAWINGS

FIG. 1 illustrates a MDR-TB real-time PCR and high resolution melt (HRM) assay algorithm according to one embodiment of the invention;

FIG. 2 illustrates representative analysis and interpretation of IS6110/rpoB portion of the assay where: (A) representative MCA for the IS6110 is shown with MTBC strains (black) contain two peaks, while NTM strains M. chelonae (-**-) and M. avium (- - -) contain only one peak each; (B) HRM graph displayed in normalized mode depicting the HRM profiles of WT strains (- - -, not visible due to overlap with solid line), A>T transversion single nucleotide polymorphisms (SNPs) (solid), and all other classes of SNP (-**-); (C) genotype difference graph with WT strains (- - -) normalized to zero where differences in strains with A>T transversion SNPs (solid) and all other classes of SNP (-**-) are shown as deviations from the WT melting pattern; and (D) positive amplification signals in crimson channel indicating the presence of an A>T transversion SNP for Asp516Val (solid);

FIG. 3 illustrates representative analysis and interpretation of katG and inhA portion of the assay where: (A) HRM plot for katG marker, shown in normalized graph mode, illustrating the HRM profile separation between WT strains (- - -) and strains containing mutations (solid; -**-); (B) Positive amplification signal in red channel indicates the presence of a G>C transversion SNP (solid); and (C) HRM plot for inhA marker displayed in normalized graph mode comparing the HRM profiles of WT strains (- - -), and strains with mutations (-**-).

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

Materials and processes are provided for a unique real-time PCR assay for the detection of mutations such as SNPs, insertions, or deletions within the Rifampicin Resistance Determinant Region (RRDR) of rpoB the detection of rifampicin (RIF) resistance, and specific regions of katG and the inhA promoter for the detection of isoniazid (INH) resistance in Mycobacterium tuberculosis (Mtb). Materials and processes are also provided to discriminate Mycobacterium tuberculosis complex (MTBC) strains from Nontuberculous Mycobacteria (NTM) strains. The current invention has utility as a composition and assay for the sensitive and rapid detection of MDR-TB, and has the potential to be modified to also detect XDR-TB and all other Mtb species both in a laboratory and field setting as well as for diagnosis of TB in a subject. The detection of Mtb in real time accelerates clinical interventions in subjects and alleviation of conditions conductive to Mtb growth.

Some embodiments combine PCR based assays with high resolution melt analysis (HRM). HRM is a molecular technique that can be used for detecting subtle genetic changes such as SNPs conferring drug resistance in Mtb. By slowly melting the DNA amplicon products of a PCR assay such as an RT-PCR assay, slight genetic differences can be visualized by changes in dissociation profiles.

The following definitional terms are used throughout the specification without regard to placement relative to these terms.

As used herein, the term “variant” defines either a naturally occurring genetic mutant of Mtb or a recombinantly prepared variation of Mtb, each of which contain one or more mutations in its genome compared to the Mtb.

As used herein, the term “derivative” in the context of a non-proteinaceous derivative defines a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated. A derivative also defined as a degenerate base mimicking a C/T mix such as that from Glen Research Corporation, Sterling, Va., illustratively LNA-dA or LNA-dT, or other nucleotide modification known in the art or otherwise. A nucleotide is optionally locked.

As used herein, the term “mutant” defines the presence of mutations in the nucleotide sequence of an organism as compared to a wild-type organism.

As used herein, the term “hybridizes under stringent conditions” describes conditions for hybridization and washing under which nucleotide sequences having at least 30%, 35%, 40%, 45%, 50%, 55%, 60%, 65%, 70%, 75%, 80%, 85%, 90%, or 95% identity to each other typically remain hybridized to each other. Such hybridization conditions are described in, for example but not limited to, Current Protocols in Molecular Biology, John Wiley & Sons, NY (1989), 6.3.1 6.3.6.; Basic Methods in Molecular Biology, Elsevier Science Publishing Co., Inc., NY (1986), pp. 75 78, and 84 87; and Molecular Cloning, Cold Spring Harbor Laboratory, NY (1982), pp. 387 389, and are well known to those skilled in the art. A non-limiting example of stringent hybridization conditions is hybridization in 6× sodium chloride/sodium citrate (SSC), 0.5% SDS at about 68° C. followed by one or more washes in 2×SSC, 0.5% SDS at room temperature. Another non-limiting example of stringent hybridization conditions is hybridization in 6×SSC at about 45° C. followed by one or more washes in 0.2×SSC, 0.1% SDS at 50 to 65° C. Other examples of stringent hybridization conditions include the other melting conditions described herein.

An “isolated” or “purified” nucleotide or oligonucleotide sequence is substantially free of cellular material or other contaminating proteins from the cell or tissue source from which the nucleotide is derived, or is substantially free of chemical precursors or other chemicals when chemically synthesized. The language “substantially free of cellular material” includes preparations of a nucleotide/oligonucleotide in which the nucleotide/oligonucleotide is separated from cellular components of the cells from which it is isolated or produced. Thus, a nucleotide/oligonucleotide that is substantially free of cellular material includes preparations of the nucleotide having less than about 30%, 20%, 10%, 5%, 2.5%, or 1% (by dry weight) of contaminating material. When nucleotide/oligonucleotide is produced by chemical synthesis, it is optionally substantially free of chemical precursors or other chemicals, i.e., it is separated from chemical precursors or other chemicals which are involved in the synthesis of the protein. Accordingly, such preparations of the nucleotide/oligonucleotide have less than about 30%, 20%, 10%, or 5% (by dry weight) of chemical precursors or compounds other than the nucleotide/oligonucleotide of interest. In some embodiments of the present invention, the nucleotide/oligonucleotide is isolated or purified.

As used herein, the term “isolated” as related to bacteria is a bacterial cell type which is separated from other organisms that are present in the natural source of the bacteria, e.g., biological material such as cells, blood, serum, plasma, saliva, urine, stool, sputum, nasopharyngeal aspirates, and so forth. The isolated bacteria are optionally used to infect a subject cell.

As used herein, the term “sample” is defined as material obtained from a biological organism, a tissue, cell, cell culture medium, or any medium suitable for mimicking biological conditions, or from the environment. Non-limiting examples include bronchoalveolar lavage fluid, bronchial aspirates, lung biopsies, post-mortem tissue specimens, sputum, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, nasal secretions, throat or nasal materials, pleural effusion, water, soil, biological waste, cell culture media, or any other fluid or solid media. In some embodiments, bacterial agents are contained in serum, whole blood, bronchoalveolar lavage fluid, bronchial aspirates, plasma, sputum, or nasal secretions.

As used herein, the term “medium” refers to any liquid or fluid biological sample in the presence or absence of bacteria. Non-limiting examples include buffered saline solution, cell culture medium, acetonitrile, trifluoroacetic acid, combinations thereof, or any other fluid recognized in the art as suitable for combination with bacteria or other cells, or for dilution of a biological sample or amplification product for analysis.

An inventive process illustratively includes exposing a sample to a forward primer and a reverse primer, wherein the forward primer and reverse primer are each specific to the RRDR of rpoB associated with rifampicin resistance, a specific region of katG or inhA promoter region associated with isoniazid resistance, or the IS6110 insert element of a Mycobacterium species. By assaying for SNPs conferring resistance and other mutations in rpoB, katG and the inhA promoter, rapid detection of MDR-TB is possible. The IS6110 insert element detection affords discrimination between Mycobacterium tuberculosis complex (MTBC) strains from Nontuberculous Mycobacteria (NTM) strains. The detection of all three amplicons (portions of rpoB, katG and inhA promoter) only occurs when M. tuberculosis complex is present in the sample and can be used for the detection of Mtb complex. As such, a process is provided that involves exposing a DNA sample to three or more primer pairs where at least one primer pair specific for at least one of rpoB, katG or inhA promoter. The presence of a single amplicon representing rpoB, katG and inhA promoter indicates the presence of Mycobacterium tuberculosis complex bacteria and excludes a nontuberculosis Mycobacteria in the sample.

The aforementioned sequences recognized by the inventive primers are similarly detectable by the inventive process. The primers are exposed to sample under conditions conducive to a polymerase chain reaction so as to yield an amplicon. The inventive process also optionally includes providing a detectable probe complementary to the amplicon under conditions allowing the probe to interact with the amplicon to allow detection of specific SNPs within the amplicon, and detecting drug resistant strains of Mtb.

As used herein, the terms “subject” and “patient” are synonymous and refer to a single or multicellular organism illustratively including, but not limited to, a human or non-human animal, optionally a mammal including a human, monkey, ape, other upper and lower primates, bovine, horse, donkey, goat, rabbit, mouse, rat, guinea pig, hamster, or non-mammals illustratively including avian species and insects, and any inclusive or other organism capable of infection or transfection by or with Mtb. It is appreciated that a subject is illustratively a single cell.

The inventive process provides a rapid, specific, and sensitive assay for detection of Mtb in samples by amplifying one or more nucleotide sequences that allow an investigator to identify and optionally to distinguish between species present in the sample by processes similar to the polymerase chain reaction (PCR). The invention is optionally used to distinguish Mtb from other NTM through assay of the IS6110 insert element and/or by the detection of all three amplicons representing a portion of rpoB, katG and the promoter region of inhA.

The present invention relates to the use of sequence information of Mtb for diagnostic processes. More particularly, the present invention provides a process for detecting the presence or absence of nucleic acid molecules of one or more Mtb species, natural or artificial variants, analogs, or derivatives thereof, in a biological sample. The process optionally involves obtaining a biological sample from one or more various sources and contacting the sample with a compound or an agent capable of detecting a nucleic acid sequence of Mtb, natural or artificial variants, analogs, or derivatives thereof, such that the presence of Mtb, natural or artificial variants, analogs, or derivatives thereof, is detected in the sample. In some embodiments, the presence of Mtb, natural or artificial variants, analogs, or derivatives thereof, is detected in the sample by a real-time polymerase chain reaction (real-time PCR or RT-PCR as used herein) using primers optionally followed by HRM analysis to identify Mtb strains optionally containing mutations at target loci. Locked nucleic acid (LNA) probes are optionally used to enhance the detection of strains containing specific SNP transversion mutations.

The present invention provides a unique real-time PCR assay for the detection of mutations conferring drug resistance in Mycobacterium tuberculosis (Mtb). The assay specifically targets the Rifampicin Resistance Determinant Region (RRDR) of rpoB for the detection of rifampicin (RIF) resistance and specific regions of katG and the inhA promoter for the detection of isoniazid (INH) resistance. Additionally, this assay is optionally multiplexed to discriminate Mycobacterium tuberculosis complex (MTBC) strains from Nontuberculous Mycobacteria (NTM) strains by targeting the IS6110 insertion element or the detection of all three amplicons representing a portion of rpoB, katG and the promoter region of inhA. High resolution melting (HRM) analysis following real-time PCR is optionally used to identify Mtb strains containing mutations at the targeted loci, and locked nucleic acid (LNA) probes are optionally used to enhance the detection of strains containing specific SNP transversion mutations. This method was used to screen 252 Mtb clinical isolates including 154 RIF resistant strains and 174 INH resistant strains, of which 148 were multidrug resistant (MDR) based on the agar proportion method of drug susceptibility testing (DST). The assay demonstrated a sensitivity and specificity of 91% and 98%, respectively, for the detection of RIF resistance, and 87% and 100% for the detection of INH resistance. Overall, this assay showed a sensitivity of 85% and a specificity of 98% for the detection of MDR strains. As such, a process optionally demonstrates sensitivity of 80% or greater, optionally 85% or greater. A process optionally demonstrates a specificity of 90% or greater, optionally from 90% to 99% or greater, optionally 91%, 92%, 93%, 94%, 95%, 96%, 97%, 98%, 99%, or greater. This method provides a rapid, robust, and inexpensive way to detect mutations that confer MDR in Mtb strains and offers several advantages over current molecular and culture-based techniques.

The present invention provides a real-time PCR assay that optionally combines HRM analysis and multiple fluorescent chemistries to detect mutations conferring drug resistance in Mtb. This assay rapidly detects the dominant mutations responsible for conferring RIF and INH resistance, and subsequently identifies MDR strains of Mtb. After screening 252 strains containing a comprehensive assortment of mutation types and combinations, sensitivity and specificity data were similar when compared to direct sequencing of the targeted loci. The assay is able to reliably detect the most common mutations associated with clinical resistance observed in the population (Table 1).

TABLE 1
Summary of assay performance for detection
of the most common mutations.

| | | # of strains | # of strains |
|---|---|---|---|
| Gene | AA change | with mutation | identified correctly |
| rpoB | Asp516Val | 9 | 9 |
| | His526Tyr | 14 | 14 |
| | Ser531Leu | 87 | 87 |
| inhA | C(−15) > T | 26 | 26 |
| promoter | | | |
| katG | Ser315Thr | 139 | 138 |

For example, the His526Tyr within the RRDR of rpoB mutation has been reported to account for up to 30% of RIF resistant strains in the USA (6). In the sample set tested, the Asp516Val, His526Tyr, and Ser531Leu accounts for 72% of the strains evaluated that are RIF resistant by DST. The assay correctly identified 100% of these strains as RIF resistant. The majority of isolates with rare mutations, and even samples known to contain mixed populations, were also detected using this assay.

Occasionally, rare mutations within rpoB were not detected. For instance, SNP transversions at locations not targeted by either LNA probe or strains with two SNPs that compensated for the deviation from the WT melting pattern were not detected (Table 2).

TABLE 2
Summary of discordant results for RTF portion of
the assay.

| | | | # of | | |
|---|---|---|---|---|---|
| | AA change | SNP | strains | HRM | DST |
| False − | Gln513Leu | CAA > CTA | 1 | S (1 of 1) | R |
| | His526Arg, | CAC > CGC, | 1 | S (1 of 1) | R |
| | Arg529Gln | CGA > CAA | | | |
| | His526Asp | CAC > GAC | 4 | S (3 of 4) | R |
| | Ser522Gln | TCG > CAG | 2 | S (1 of 2) | R |
| | Ser531Trp | TCG > TGG | 2 | S (2 of 2) | R |
| | WT | WT | 6 | S (6 of 6) | R |
| False + | Leu511Pro | CTG > CCG | 1 | R (1 of 1) | S |
| | Leu533Pro | CTG > CCG | 3 | R (3 of 3) | S |

S = strain predicted as susceptible by given method
R = strain predicted as resistant by given method

Because Class III and IV SNP changes involve bases exchanges that do not change the number of hydrogen bonds between the bases, HRM alone was not sufficient to detect these SNPs and this remains a limitation of this technique for use in diagnostics. However, to circumvent this potential shortcoming, the present invention includes probes, optionally LNA probes, designed to target the most common SNP transversions that would have otherwise been missed. As expected, when the primers and probes of SEQ ID NOs. 1-17 are used, the assay is not predictive of resistance in cases where mutations conferring resistance are outside the RRDR or strains that contained no mutation in rpoB but were determined to be RIF resistant by DST (6 of 252 strains) (Table 2). Similarly, INH resistance occurred in strains containing no mutations in the targeted loci, yet resistant by DST (22 of 252 strains). These strains are likely to be resistant by other mechanisms or by unknown mechanisms of INH resistance, and this possibility has been previously discussed (15). It is appreciated, however, that the inventive processes are equally applicable to detection of these and other types of resistance by one of skill in the art with knowledge of the sequence mutation conferring the resistant characteristics. As such, the inventive processes are not limited to detection of the specific mutations highlighted herein, but are equally applicable to detection of other mutations in the same or alternate regions of the Mtb genome.

When compared to conventional HRM assays that have been developed for the rapid diagnosis of drug resistance in TB (5, 12), an inventive assay is more robust and informative, providing additional critical details about strains. First, some embodiments of an inventive assay discern MTBC from NTM strains. This is useful in a clinical setting where patients, especially those who are immuno-compromised, could possibly be infected with NTM. Additionally, other HRM assays rely solely on the identification of SNPs associated with RIF resistance as a predictor for MDR. While up to 97% of RIF strains have in fact been determined to be MDR, mono-RIF resistance is possible, albeit rare (19). The lack of information provided by other assays regarding INH susceptibility remains a shortcoming and could incorrectly suggest that INH would not be effective if included in a drug treatment regimen. By using RIF resistance as the sole predictor for MDR, it potentially removes INH from drug regimens where it would be more effective and have fewer side effects than other drugs. An inventive assay described herein will optionally detect resistance to both RIF and INH simultaneously or sequentially, which offers a significant improvement over current comparable diagnostic methods.

The advantages of the inventive assay over current molecular or culture-based techniques are numerous. This comprehensive assay rapidly provides a significant amount of information at a much lower cost than culture-based or sequencing methods. Within approximately five hours of obtaining DNA, the assay is able to confirm the sample as MTBC, determine the RIF and INH resistance pattern, and therefore, provide a preliminary MDR diagnosis. In some embodiments, the quick turnaround time is due, in part, to the fact that the methods have been optimized so that the same PCR conditions are used for all portions. Comparable assays require that PCR conditions be modified for each target loci (11), or sometimes require the use of multiple platforms (2). The inventive assay is compatible with several methods of DNA preparation and is valid over at least a three-log range of DNA concentrations, eliminating the need for time-consuming standardized DNA extractions and quantification. The assay detects SNP transversion mutations with LNA probes rather than resorting to the commonly used, yet cumbersome, approach of spiking the sample with secondary control DNA (5). As a result, sample tubes remain closed between real-time PCR and HRM steps, minimizing the chance for contamination. The use of LNA probes to detect certain SNP transversions greatly reduces user subjectivity and increases confidence in the results. In some embodiments, the use of several real-time PCR chemistries in one tube is fostered by the multi-channel format of the Qiagen Rotor-Gene 6000 platform or other platform that combines multiple fluorescent chemistries together in one reaction to achieve maximum discrimination.

An additional appealing feature of an inventive assay is the flexibility it allows the user. Components can be adjusted to perform the assay with or without specific markers, providing countless options as to how to meet the needs of the laboratory. Because of the molecular basis of the techniques, real-time PCR and HRM analysis cannot detect drug resistance conferred by unknown mutations or by unknown mechanisms, and this limitation is common among any sequence-dependant detection method. However, as new mutations are identified or become more prevalent in a population, additional probes are easily designed by software or by hand and added to an inventive assay to update an inventive assay. This adaptability is an important feature because patterns of drug resistance can vary widely based on the drugs used in a particular geographic area (14).

A significant application of an inventive assay is for improving TB treatment. Currently in the USA, the standard empiric treatment for adults who have not previously received TB therapy consists of four drugs: RIF, INH, ethambutol (EMB), and pyrazinamide (PZA) until DST results are available (1). It is worth noting, however, that in 2008, 8.2% of previously untreated TB patients were infected with strains that were INH resistant (3). Because of this relatively high frequency of INH resistance, EMB is included in the treatment regimen in order to prevent the emergence of RIF resistance (1). However, because of the added cost and potential side effects of EMB (4), such as optic neuritis, it is preferable to omit it from the treatment regimen when its use is not necessary. In this case, the inventive assay is useful not only to predict or identify drug resistance, but also to predict susceptibility, thereby identifying potentially more desirable and effective drugs that could be added to enhance treatment, while eliminating those with harsher side effects that are not as clinically effective. Additionally, by using an inventive assay as a preliminary indicator for drug resistance, those strains that give positive results can be fast tracked for second-line DST, as opposed to waiting several weeks to obtain first-line DST results. Taken together, these applications illustrate the clinical value of the assay.

Mtb is detected by culture, urine antigen test, seroconversion, real-time polymerase chain reaction (PCR), other nucleic acid based assays, mass spectrometry, high pressure liquid chromatography (HPLC), or PCR detection such as by amplification of genetic sequences by primers of SEQ ID NOS.

Also, a diagnostic assay process is provided for detection of Mtb infection in a patient wherein a clinical sample from a patient suspected of being infected with Mtb is exposed to a forward primer and a reverse primer for specific amplification and detection of a region of rpoB, katG or inhA promoter.

The inventive processes are amenable to provide a preliminary diagnosis of MDR-TB in a subject.

The term “nucleotide” is intended to mean a base-sugar-phosphate combination either natural or synthetic, linear, circular and sequential arrays of nucleotides and nucleosides, e.g. cDNA, genomic DNA, RNA, oligonucleotides, oligonucleosides, and derivatives thereof.

Included in this definition are modified nucleotides which include additions to the sugar-phosphate groups or to the bases.

A sample is optionally a fluidic sample. Illustratively, a fluidic sample such as serum or cell lysate is diluted in a buffered saline solution suitable for assay of a Mtb species. Alternatively, a sample is solid wherein a suspension is created in a buffered saline solution or the solid is dissolved in a solvent such as lysis buffer. An illustrative example of operative buffered solutions are 50 mM Tris-HCl, 10 mM MgCl₂, 100 mM NaCl, pH 8.0 or 25 mM Tris/HCl, pH 7.6, 25 mM KCl, 5 mM MgCl₂. It is appreciated that other buffered or non-buffered solutions are similarly operable. Other buffers illustratively include HEPES, Tris, phosphate, carbonate, imidizole, acetate, or any other buffer known in the art. Salts and other cations are further operable in the invention. (See e.g. Endo, Y, et al., J Biol Chem, 1987; 262:8128-30.) Magnesium ions are optionally included in a buffer or solution. Endo, Y, J Biol Chem, 1988; 263:8735-8739. Magnesium is optionally present between 5 and 15 mM.

The inventive process includes a polymerization reaction. The polymerization reaction is performed by a nucleic acid polymerizing enzyme that is illustratively a DNA polymerase, RNA polymerase, reverse transcriptase, mixtures thereof, or other polymerases known in the art. It is further appreciated that accessory proteins or molecules are present to form the replication machinery. The process of the present invention optionally involves a real-time PCR assay that is optionally quantitative.

The assays are performed on an instrument designed to perform polymerization reactions and optionally HRM, for example those available from Applied Biosystems (Foster City, Calif.). In specific embodiments, the present invention provides a real-time quantitative PCR assay to detect the presence of one or more Mtb species, natural or artificial variants, analogs, or derivatives thereof, in a biological sample by subjecting the Mtb nucleic acid from the sample to PCR reactions using specific primers, and detecting the amplified product using a probe. In some embodiments, the probe includes one or more LNAs and which consists of an oligonucleotide with a 5′-reporter dye and a 3′-quencher dye.

Optionally, a fluorescent reporter dye, such as FAM dye (illustratively 6-carboxyfluorescein), is covalently linked to the 5′ end of the oligonucleotide probe. Other dyes illustratively include TAMRA, AlexaFluor dyes such as AlexaFluor 495 or 590, Cascade Blue, Marina Blue, Pacific Blue, Oregon Green, Rhodamine, Fluoroscein, TET, HEX, Cy5, Cy3, Quasar670, and Tetramethylrhodamine. Each of the reporters is optionally quenched by a dye at the 3′ end or other non-fluorescent quencher. Quenching molecules are suitably matched to the fluorescence maximum of the dye. Any suitable fluorescent probe for use in real-time PCR detection systems is illustratively operable in the invention. Similarly, any quenching molecule for use in real-time PCR systems is illustratively operable. In some embodiments a 6-carboxyfluorescein reporter dye is present at the 5′-end and matched to Black Hole Quencher (BHQ1, Biosearch Technologies, Inc., Novato, Calif.). The fluorescence signals from these reactions are captured at the end of extension steps as PCR product is generated over a range of the thermal cycles, thereby allowing the quantitative determination of the bacterial load in the sample based on an amplification plot.

It is appreciated that other detection systems, techniques, and labels are operative herein. Illustratively, a probe is labeled with a radioactive marker. Illustrative radioactive labels include ³H, ¹³C, ³²P, ¹²⁵I, ¹³¹I, ²²Na, ⁵¹Cr, and other radioactive labels known in the art.

The Mtb nucleic acid sequences are optionally amplified before being detected. The term “amplified” defines the process of making multiple copies of the nucleic acid from a single or lower copy number of nucleic acid sequence molecule. The amplification of nucleic acid sequences is optionally carried out in vitro by biochemical processes known to those of skill in the art. The amplification agent is optionally any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, AmpliTaq Gold DNA Polymerase from Applied Biosystems, other available DNA polymerases, reverse transcriptase (preferably iScript RNase H+ reverse transcriptase), ligase, and other enzymes, including heat-stable enzymes (i.e., those enzymes that perform primer extension after being subjected to temperatures sufficiently elevated to cause denaturation). Suitable enzymes will facilitate combination of the nucleotides in the proper manner to form the primer extension products that are complementary to each mutant nucleotide strand. Generally, the synthesis is initiated at the 3′-end of each primer and proceed in the 5′-direction along the template strand, until synthesis terminates, producing molecules of different lengths. It is appreciated that amplification agents that initiate synthesis at the 5′-end and proceed in the other direction, using the same process as described above are similarly operable. In any event, the process of the invention is not to be limited to the embodiments of amplification described herein.

One process of in vitro amplification optionally used according to this invention is the polymerase chain reaction (PCR) described in U.S. Pat. Nos. 4,683,202 and 4,683,195. The term “polymerase chain reaction” refers to a process for amplifying a DNA base sequence using a heat-stable DNA polymerase and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. Many polymerase chain processes are known to those of skill in the art and are optionally used in the process of the invention. For example, DNA is optionally subjected to 30 to 35 cycles of amplification in a thermocycler as follows: 95° C. for 30 sec, 52 to 60° C. for 1 min, and 72° C. for 1 min, with a final extension step of 72° C. for 5 min. For another example, DNA is subjected to 35 polymerase chain reaction cycles in a thermocycler at a denaturing temperature of 95° C. for 30 sec, followed by varying annealing temperatures ranging from 54 to 58° C. for 1 min, an extension step at 70° C. for 1 min, with a final extension step at 70° C. for 5 min. The parameters of PCR cycling times and number of steps are dependent on the primer pair, their melting temperature, and other considerations obvious to those known in the art. It is appreciated that optimizing PCR parameters for various probe sets is well within the skill of the art and is performed as mere routine optimization.

The primers or probes for use for amplifying the nucleic acid sequences of Mtb are illustratively prepared using any suitable process, such as conventional phosphotriester and phosphodiester processes or automated embodiments thereof so long as the primers or probes are capable of hybridizing to the nucleic acid sequences of interest. One process for synthesizing oligonucleotides on a modified solid support is described in U.S. Pat. No. 4,458,066. The exact length of primer or probe will depend on many factors, including temperature, buffer, and nucleotide composition. The primer generally primes the synthesis of extension products in the presence of the inducing agent for amplification.

Primers used according to the process of the invention are complementary to each strand of nucleotide sequence to be amplified. The term “complementary” means that the primers hybridize with their respective strands under conditions that allow the agent for polymerization to function. In other words, the primers hybridize with Mtb sequences(s) and permit amplification of the nucleotide sequence. Optionally, the 3′ terminus of the primer that is extended is perfectly base paired with the complementary flanking strand. Optionally, probes possess nucleotide sequences complementary to one or more strands of the amplification product. Optionally, the primers and probes are complementary to genetic sequences specific to one of the region of rpoB, katG or inhA promoter. Optionally, primers contain the nucleotide sequences of one of the herein detailed SEQ ID NOS. It is appreciated that the complement of the aforementioned primer and probe sequences are similarly suitable for use in the invention. It is further appreciated that oligonucleotide sequences that hybridize with the inventive primer and probes are also similarly suitable. Multiple positions are available for hybridization on the Mtb genome and will be also suitable for hybridization with a probe when used with the proper forward and reverse primers.

Those of ordinary skill in the art will know of various amplification processes that are optionally utilized to increase the copy number of target nucleic acid sequence. The nucleic acid sequences detected in the process of the invention are optionally further evaluated, detected, cloned, sequenced, and the like, either in solution or after binding to a solid support, by any process usually applied to the detection of a specific nucleic acid sequence such as another polymerase chain reaction, oligomer restriction (Saiki et al., BioTechnology 3:1008 1012 (1985)), allele-specific oligonucleotide (ASO) probe analysis (Conner et al., PNAS 80: 278 (1983)), oligonucleotide ligation assays (OLAs) (Landegren et al., Science 241:1077 (1988)), RNase Protection Assay and the like. Molecular techniques for DNA analysis have been reviewed (Landegren et al., Science 242:229 237 (1988)). Following DNA amplification, the reaction product is optionally detected by Southern blot analysis, with or without using radioactive probes. In such a process, for example, a small sample of DNA containing the nucleic acid sequence obtained from the tissue or subject is amplified, and analyzed via a Southern blotting technique. The use of non-radioactive probes or labels is facilitated by the high level of the amplified signal. In one embodiment of the invention, one nucleoside triphosphate is radioactively labeled, thereby allowing direct visualization of the amplification product by autoradiography. In another embodiment, amplification primers are fluorescently labeled and run through an electrophoresis system. Visualization of amplified products is by laser detection followed by computer assisted graphic display, without a radioactive signal.

Other methods of detecting amplified oligonucleotide illustratively include gel electrophoresis, mass spectrometry, liquid chromatography, fluorescence, luminescence, gel mobility shift assay, fluorescence resonance energy transfer, nucleotide sequencing, enzyme-linked immunoadsorbent assay, high performance liquid chromatography, ultra-high performance liquid chromatography, enzyme-linked immunoadsorbent assay, real-time PCR, affinity chromatography, immunoenzymatic methods (Ortiz, A and Ritter, E, Nucleic Acids Res., 1996; 24:3280-3281), streptavidin-conjugated enzymes, DNA branch migration (Lishanski, A, et al., Nucleic Acids Res., 2000; 28(9):e42), enzyme digestion (U.S. Pat. No. 5,580,730), colorimetric methods (Lee, K., Biotechnology Letters, 2003; 25:1739-1742), or combinations thereof.

The term “labeled” with regard to the probe is intended to encompass direct labeling of the probe by coupling (i.e., physically linking) a detectable substance to the probe, as well as indirect labeling of the probe by reactivity with another reagent that is directly labeled. Examples of indirect labeling include detection of a probe using a fluorescently labeled antibody and end-labeling or centrally labeling of a DNA probe with biotin such that it can be detected with fluorescently labeled streptavidin. The detection method of the invention is optionally used to detect RNA (particularly mRNA) or genomic nucleic acid in a sample in vitro as well as in vivo. For example, in vitro techniques for detection of nucleic acid include northern hybridizations, in situ hybridizations, RT-PCR, real-time PCR, and DNase protection.

The size of the primers used to amplify a portion of the nucleic acid sequence of MtB is optionally at least 5, and often 10, 15, 20, 25, or 30 nucleotides in length. Optionally, the GC ratio is above 30%, 35%, 40%, 45%, 50%, 55%, or 60% so as to prevent hair-pin structure on the primer. Furthermore, the amplicon is optionally sufficiently long enough to be detected by standard molecular biology methodologies. The forward primer is optionally longer than the reverse primer. Techniques for modifying the Tₘ of either primer are operable herein. An illustrative forward or reverse primer or probe contains LNA-dA, LNA-dC, LNA-dG, and LNA-dT (Glen Research Corporation) optionally to match Tₘ with a corresponding alternate primer or to provide a probe that will provide a specific melt profile with a transversion mutation of interest.

Accuracy of the base pairing of DNA sequencing is provided by the specificity of the enzyme. Error rates for Taq polymerase tend to be false base incorporation of 10⁻⁵ or less. (Johnson, Annual Reviews of Biochemistry, 1993: 62:685-713; Kunkel, Journal of Biological Chemistry, 1992; 267:18251-18254). Specific examples of thermostable polymerases illustratively include those isolated from Thermus aquaticus, Thermus thermophilus, Pyrococcus woesei, Pyrococcus furiosus, Thermococcus litoralis and Thermotoga maritima. Thermodegradable polymerases illustratively include E. coli DNA polymerase, the Klenow fragment of E. coli DNA polymerase, T4 DNA polymerase, T7 DNA polymerase and other examples known in the art. It is recognized in the art that other polymerizing enzymes are similarly suitable illustratively including E. coli, T7, T3, SP6 RNA polymerases and AMV, M-MLV, and HIV reverse transcriptases.

The polymerases are optionally bound to the primer. When the genetic material of Mtb is a single-stranded DNA molecule due to heat denaturing the polymerase is bound at the primed end of the single-stranded nucleic acid at an origin of replication. A binding site for a suitable polymerase is optionally created by an accessory protein or by any primed single-stranded nucleic acid.

It is further appreciated that the proteinaceous material of the polymerization enzyme in the case of a DNA polymerase is optionally immobilized on a solid support surface either reversibly or irreversibly. For example, RNA polymerase was successfully immobilized on an activated surface without loss of catalytic activity. Yin et al., Science, 1995; 270: 1653-57. Alternatively, an antibody antigen pair is utilized to bind a polymerase enzyme to a support surface whereby the support surface is coated with an antibody that recognizes an epitope on the protein antigen. When the antigen is introduced into the reaction chamber it is reversibly bound to the antibody and immobilized on the support surface. A lack of interference with catalytic activity in such a process has been reported for HIV reverse transcriptase. Lennerstrand, Analytical Biochemistry, 1996; 235:141-152. Additionally, DNA polymerase immobilization has been reported as a functional immobilization process in Korlach et al., U.S. Pat. No. 7,033,764 B2. Finally, any protein component is optionally biotinylated such that, illustratively, a biotin streptavidin interaction is created between the support surface and the target immobilized antigen.

A real-time PCR assay system is employed such as the TAQMAN system available from Applied Biosystems (Foster City, Calif.) or the iCycler iQ real-time detection system (Bio-Rad, Hercules, Calif.). It is appreciated that a probe based process, intercalator-based process, or other process known in the art are operable herein. Suitable probes target the amplicon region of Mtb and are optionally between 15 and 60 nucleotides long, are unique to the target sequence, are not prone to dimerization, and do not possess repeat regions. Processes of probe design and considerations for use are recognized in the art.

In a further embodiment detection of PCR products is achieved by mass spectrometry. Mass spectrometers are prevalent in the clinical laboratory. Similar to fluorescence based detection systems mass spectrometry is capable of simultaneously detecting multiple amplification products for a multiplexed and controlled approach to accurately quantifying components of biological or environmental samples.

Multiple mass spectrometry platforms are suitable for use in the invention illustratively including matrix assisted laser desorption ionization time of flight mass spectrometry (MALDI), electrospray mass spectrometry, electrospray ionization-Fourier transform ion cyclotron resonance mass spectrometry (ESI-FTICR), multi-stage mass spectrometry fragmentation analysis (MS/MS), mass spectrometry coupled with liquid chromatography such as high performance liquid chromatography mass spectrometry (HPLC) and ultra performance liquid chromatography isotope dilution tandem mass spectrometry (UPLC-ID/MS/MS), and variations thereof.

Optionally, multiple amplification products are simultaneously produced in a PCR reaction that are then available for simultaneous detection and quantification. Thus, multiple detection signals are inherently produced or emitted that are separately and uniquely detected in one or more detection systems. It is appreciated that multiple detection signals are optionally produced in parallel. Optionally, a single biological sample is subjected to analysis for the simultaneous or sequential detection of Mtb genetic sequences. It is appreciated that two or more independent or overlapping sequences are simultaneously or sequentially measured in the instant inventive process. Oligonucleotide matched primers (illustratively SEQ ID NOS: 1 and 2) are simultaneously or sequentially added and the biological sample is subjected to proper thermocycling reaction parameters. For detection by mass spectrometry, a single sample of the amplification products from each gene is simultaneously analyzed allowing for rapid and accurate determination of the presence of Mtb. Optionally, analysis by real-time PCR is employed capitalizing on multiple probes with unique fluorescent signatures. Thus, each gene or other genetic sequence is detected without interference by other amplification products. This multi-target approach increases confidence in quantification and provides for additional internal control.

The invention also encompasses a kit for detecting the presence of mutations in a Mtb DNA sample. The kit, for example, includes oligonucleotides capable of detecting mutations conferring MDR-TB in a test DNA sample and discrimination of MTBC from NTM strains of mycobacteria.

For oligonucleotide-based kits, the kit includes, for example: (1) a pair of primers (one forward and one reverse) useful for amplifying a nucleic acid amplicon synthesized in the presence of Mtb DNA; and/or optionally (2) a probe operable for detecting certain transversion SNPs within the amplicon generated from Mtb DNA. The kit also optionally includes a buffering agent, a preservative, or a protein stabilizing agent. It is appreciated that a kit is optionally as simple as a primer or probe for addition to a sample or as complex as all reagents, enzymes, oligonucleotides, and detection apparatus necessary for full detection and quantification of targeted Mtb DNA amplicons in a sample. The kit optionally includes components necessary for detecting the detectable agent (e.g., synthesized amplicon). The kit also optionally contains a control sample DNA or a series of control sample DNAs that is assayed and compared to the test sample DNA. Each component of the kit is optionally enclosed within an individual container(s) and all of the various containers are optionally enclosed within a single package along with instructions for use.

Ancillary reagents are any signal producing system materials for detection of Mtb in any suitable detection process such as real-time PCR, ELISA, mass spectrometry, Southern blot, immunoprecipitation, HPLC, UHPLC, or other process known in the art. In embodiments, a kit optionally includes a microtiter plate or other support or chamber such as an collection tube sealable or not sealable, control sample containing Mtb, buffer, swab or other sample collection devices, control reagents such as competing or unlabelled reagents, control substrate and relevant primers and probes, and other materials and reagents for detection. The kit optionally includes instructions printed or in electronic form and customer support contact information. Probes in a signal producing system or otherwise are optionally labeled with a fluorophore, biotin, peroxidase, or other enzymatic or non-enzymatic detection label such as a radioactive label or otherwise.

The components of the kit are any of the reagents described above or other necessary and non-necessary reagents known in the art for solubilization, detection, washing, storage, or other need for in an assay kit.

Methods involving conventional biological techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; Current Protocols in Molecular Biology, ed. Ausubel et al., Greene Publishing and Wiley-Interscience, New York, 1992 (with periodic updates); and Short Protocols in Molecular Biology, ed. Ausubel et al., 52 ed., Wiley-Interscience, New York, 2002.

Various aspects of the present invention are illustrated by the following non-limiting examples. Additional examples can be found in Ramirez et al., J Clin Microbiol. 2010; 48(11):4003-9. The examples are for illustrative purposes and are not a limitation on any practice of the present invention. It is understood that variations and modifications can be made without departing from the spirit and scope of the invention.

### Example 1

### Bacterial Strains, Growth Condition, and Drug Susceptibility Testing

Clinical Mtb strains used in this study are obtained from the culture collection at the Mycobacteriology Laboratory Branch, CDC. Information regarding patients linked to the clinical isolates used in this study is protected as described in a protocol approved by the CDC Institutional Review Board. Drug susceptibility testing (DST) is performed using the agar proportion method previously described and in accordance with the Clinical and Laboratory Standards Institute (10). Susceptibility is tested at 1 μg/ml for RIF and 0.2, 1.0, and 5.0 μg/ml for INH.

### Example 2

### DNA Preparation and Sequencing of Regions of rpoB, katG, and the inhA Promoter

DNA is prepared from Mtb cultures grown at 37° C. that had reached saturation using the previously described Fast Prep method (13). Amplicons are generated by PCR for regions of rpoB (rpoB-F 5′CTTGCACGAGGGTCAGACCA (SEQ ID NO: 1) and rpoB-R 5′ATCTCGTCGCTAACCACGCC (SEQ ID NO: 2),) katG (katG-F 5′AACGACGTCGAAACAGCGGC (SEQ ID NO: 3) and katG-R 5′GCGAACTCGTCGGCCAATTC (SEQ ID NO: 4)), and the inhA promoter (inhA-F 5′TGCCCAGAAAGGGATCCGTCATG (SEQ ID NO: 5) and inhA-R 5′ATGAGGAATGCGTCCGCGGA (SEQ ID NO: 6)). Amplicons are treated with ExoSAP-IT (Affymetrix, Inc.) according to the manufacturer's instructions and diluted 1:10 for sequencing. The sequencing reactions for the treated amplicons contain BigDye Terminator v3.1 mix and the BigDye 5× Sequencing Buffer (Applied Biosystems) with the same primers used for PCR. Sequencing is performed using the ABI 3130XL Genetic Analyzer. Sequence analysis is performed using ABI Sequence Analysis software and aligned using DNASTAR Lasergene 8.0.

### Example 3

### Real-Time PCR

Primers and probes for the real-time PCR assays are designed to target four different locations within the Mtb genome. The primers and probes targeting the rpoB gene and the IS6110 insertion element are combined into a SYBR based duplex assay. The primers for rpoB are manually designed to selectively amplify a 152 by amplicon spanning the RRDR (rpoB-F, 5′GCCGCGATCAAGGAGTTCT (SEQ ID NO: 7) and rpoB-R 5′-ACGTCGCGGACCTCCAG (SEQ ID NO: 8)). The primers for the IS6U0 insertion element are manually designed to generate a 179 by amplicon within the MTBC-specific region of IS6110 (IS6110-F 5′CCACCATACGGATAGGGGA (SEQ ID NO: 9) and IS6110-R 5′TGGACCGCCAGGGCT (SEQ ID NO: 10)). In order to identify SNP transversions within rpoB, two locked nucleic acid (LNA) probes are included in this assay. Beacon Designer software (PREMIER Biosoft) is used to design two LNA probes targeting specific loci within the rpoB amplicon to detect A>T SNP changes in specific strains at Asp516 and His526, respectively (rpoB LNA D516V 5′aattcaTggTccAgaAcaa (SEQ ID NO: 11) and rpoB LNA H526L, 5′gtTgaCccTcaAgc (SEQ ID NO: 12): capital letters indicate a locked base). Each LNA probe is designed with a unique 5′ fluorescent label, with Quasar 705 on the LNA D516V probe, and CalR610 on the LNA H526L probe. Both are quenched on the 3′ end with BHQ2. RRDR mutant strains are provided in Table 3.

TABLE 3
Summary of strains containing mutations within the RRDR of rpoB¹

| AA change | SNP | # of strains |
|---|---|---|
| Asp516Ala | GAC > GCC | 1 |
| Asp516Tyr | GAC > TAC | 2 |
| Asp516Tyr, Ser531Leu | GAC > TAC, TCG > TTG | 1 |
| Asp516Val | GAC > GTC | 9 |
| Phe586Val, Asp516Val | TTC > GTC, GAC > GTC | 1 |
| insertion at nt 1295 | GCC insertion | 2 |
| Gln490Arg | CAG > CGG | 1 |
| Gln513His, Leu533Pro | CAA > CAC, CTG > CCG | 3 |
| Gln513Leu | CAA > CTA | 1 |
| His526Arg | CAC > CGC | 3 |
| His526Arg, Arg529Gln | CAC > CGC, CGA > CAA | 1 |
| His526Asn, Ser531Leu | CAC > AAC, TCG > TTG | 1 |
| His526Asp | CAC > GAC | 4 |
| His526Gln, Leu533Pro | CAC > CAG, CTG > CCG | 1 |
| His526Gly | CAC > GGC | 1 |
| His526Leu | CAC > CTC | 2 |
| His526Tyr | CAC > TAC | 14 |
| Ile572Leu, Asp516Gly | ATC > CTC, GAC > GGC | 1 |
| Leu459Arg, Asp516Tyr | CTG > CGG, GAC > TAC | 1 |
| Leu459Arg, His526Tyr | CTG > CGG, CAC > TAC | 1 |
| Leu511Pro | CTG > CCG | 1 |
| Leu533Pro | CTG > CCG | 3 |
| Phe514Phe, Ser531Leu | TTC > TTT, TCG > TTG | 1 |
| Ser522Gln | TCG > CAG | 2 |
| Ser531Leu | TCG > TTG | 87 |
| Ser531Leu, His526Tyr | TCG > TTG, CAC > TAC | 1 |
| Ser531Leu, ne480Val | TCG > TTG, ATC > GTC | 1 |
| Ser531Phe | TCG > TTC | 1 |
| Ser531Trp | TCG > TGG | 2 |
| Thr481Ala, Ser531Leu | ACC > GCC, TCG > TTG | 1 |
| WT | WT | 101 |

¹Of the 252 strains analyzed, 151 contained mutations within the RRDR of rpoB.

The katG and inhA promoter mutations are targeted independently in singleplex assays. The primers are designed to span a region within each gene known to contain mutations conferring INH resistance. The primers for the katG gene are generated using Beacon Designer software (PREMIER Biosoft) and produce an 123 by amplicon (katG-F 5′TCGTATGGCACCGGAACC (SEQ ID NO: 13) and katG-R 5′CAGCTCCCACTCGTAGCC (SEQ ID NO: 14)). The software is also used to design an LNA probe to bind within the amplicon and detect the Ser315Thr mutation, a G>C SNP change found in resistant strains (katG LNA S315T, 5′atcaCcaCcgGcaTcg (SEQ ID NO: 15): capital letters indicate a locked base). The LNA S315T probe is labeled on the 5′ end with Q670 and quenched with BHQ3 on the 3′ end. The primers for the inhA promoter region are designed manually, and generate a 132 by amplicon (inhA-F 5′CGTTACGCTCGTGGACATAC (SEQ ID NO: 16) and inhA-R 5′GTTTCCTCCGGTAACCAGG (SEQ ID NO: 17)). Strain mutants of inhA promoter or katG identified are provided in Table 4.

TABLE 4
Summary of strains containing mutations in the
inhA promoter or katG¹

| Gene | AA change | SNP | # of strains |
|---|---|---|---|
| inhA promoter | C(−15) > T | C > T | 26 |
| | T(−8) > C | T > C | 3 |
| katG | Gly273Ser | GGT > AGT | 1 |
| | Ser315Asn | AGC > AAC | 1 |
| | Ser315He | AGC > ATC | 1 |
| | Ser315Thr | AGC > ACC | 139 |
| | Ser315Thr, | AGC > ACC, | |
| | Ile335Val | ATC > GTC | 2 |
| | WT | WT | 97 |

¹Of the 252 strains analyzed, 173 contained mutations in the inhA promoter, katG, or both.

The SYBR reaction mixture is prepared using the Universal SYBR GreenER qPCR kit (Invitrogen), containing the following components per reaction in all three assays: 12.5 μL of 2× master mix, 5 μL of the template, and nuclease-free water (Promega), for a total reaction volume of 25 μL. Primers and probes are added to the appropriate master mix in the following concentrations: 100 nM rpoB, IS6110, katG, and inhA primers; 250 nM katG LNA probe; 500 nM rpoB LNA516 probe; 1 μM rpoB LNA526 probe. Amplification is performed on the Rotor-Gene 6000 system (Qiagen) using the following conditions: 1 cycle of 95° C. for 2 min, followed by 45 cycles of 95° C. for 15 s and 63° C. for 30 s, with data acquired on the 63° C. step in the green, orange, red, and crimson channels. Following amplification, HRM is performed between 80° C. and 89° C. at a rate of 0.02° C. per step. All samples were tested in duplicate. All control and unknown DNA samples are diluted such that Ct values fell between cycles 19-29.

### Example 4

### Melt Curve Data Analysis

Melt curves are generated by taking the derivative of the raw fluorescence level during each HRM step. The dF/dT plot contains specific peaks at the melting temperature of the double stranded products.

### Example 5

### HRM Data Analysis

The HRM curves are analyzed by selecting two normalization regions, one occurring prior to the melting of the amplicon and one following complete separation of the two strands. The normalization regions utilized for each assay are as follows: rpoBHS6110 assay, ˜85° C. to 85.5° C. (region one) and ˜88° C. to 88.5° C. (region two); katG assay, ˜80° C. to 80.5° C. (region one) and ˜83.5° C. to 84° C. (region two); inhA assay, ˜81.5° C. to 82° C. (region one) and ˜85° C. to 85.5° C. (region two). HRM curves are viewed in “replicate mode”, a user option whereby a single melt curve is derived by averaging all of the replicates for that sample. The “difference graph” is generated by normalizing the HRM profile of the wild type (WT) control strain to zero, and highlighting any deviations (i.e., resistant isolates) as distinct curves.

### Example 6

### Strain Analysis Workflow and Analysis

The rpoB/156110 and katG/inhA assays are used to screen a panel of DNA from 252 Mtb clinical isolates obtained from the Mycobacterial Laboratory Branch. The panel includes 29 different types or combinations of mutations within rpoB, 5 unique types or combination of mutations within katG, and 2 distinct types or combination of mutations within the promoter region of inhA. The results from the assay are compared to the phenotypic DST and sequencing results for the targeted loci.

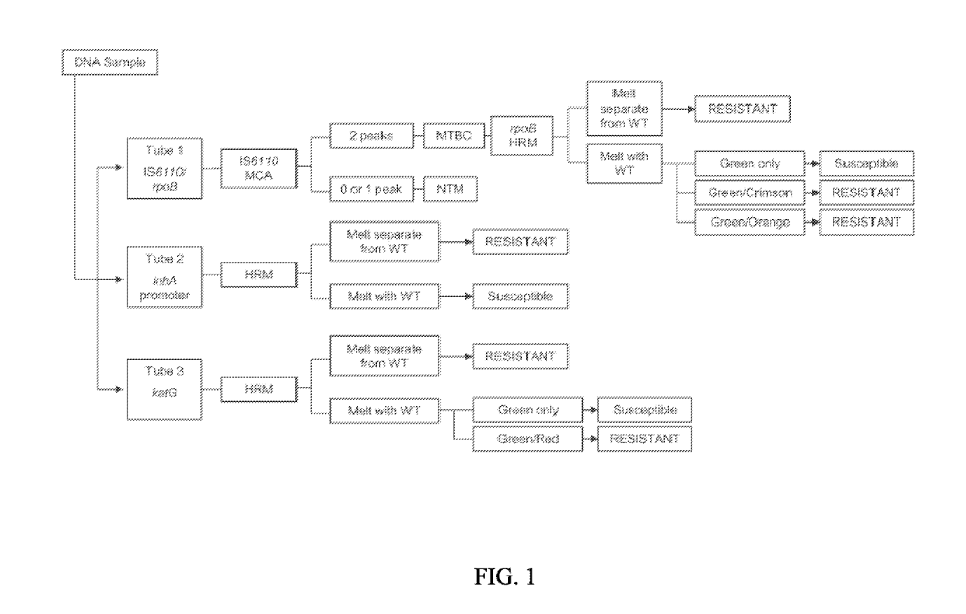

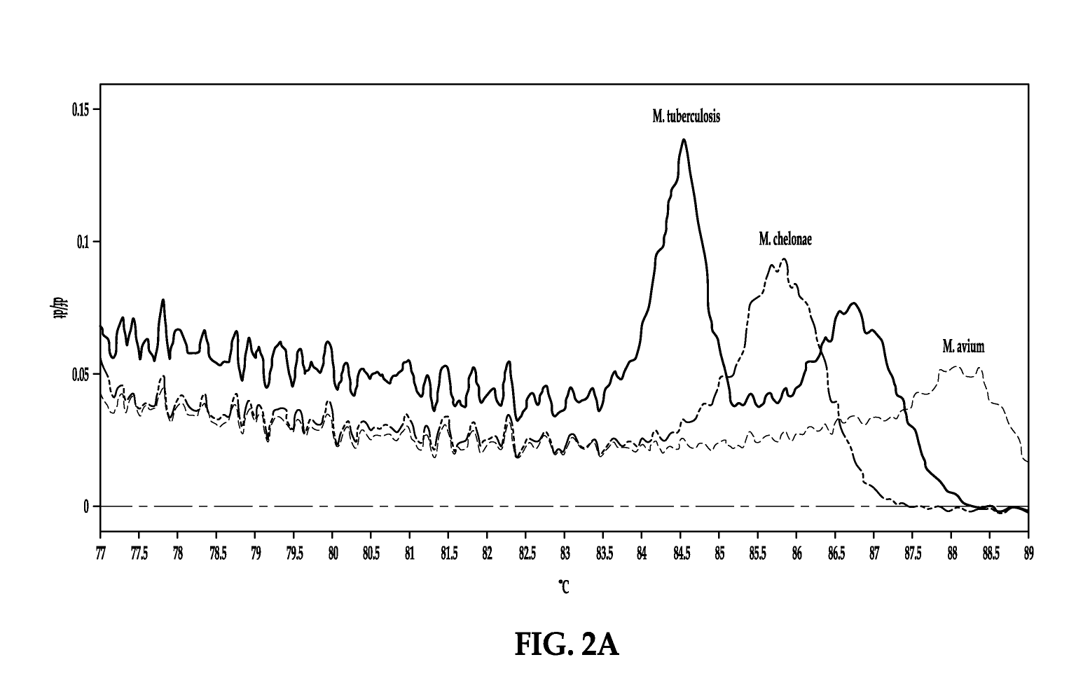

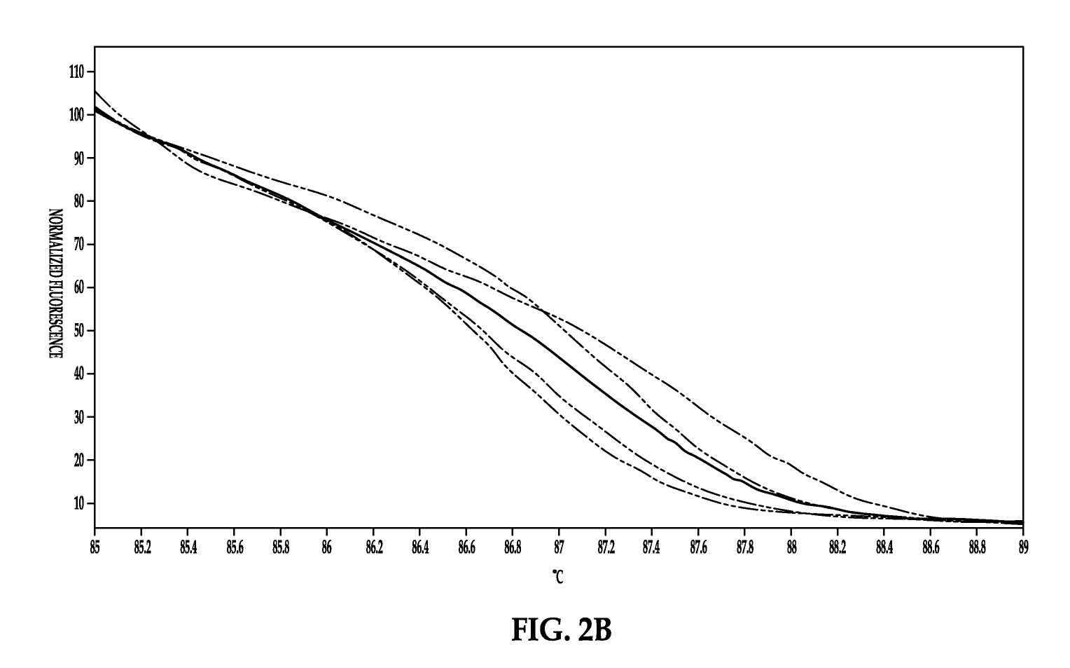

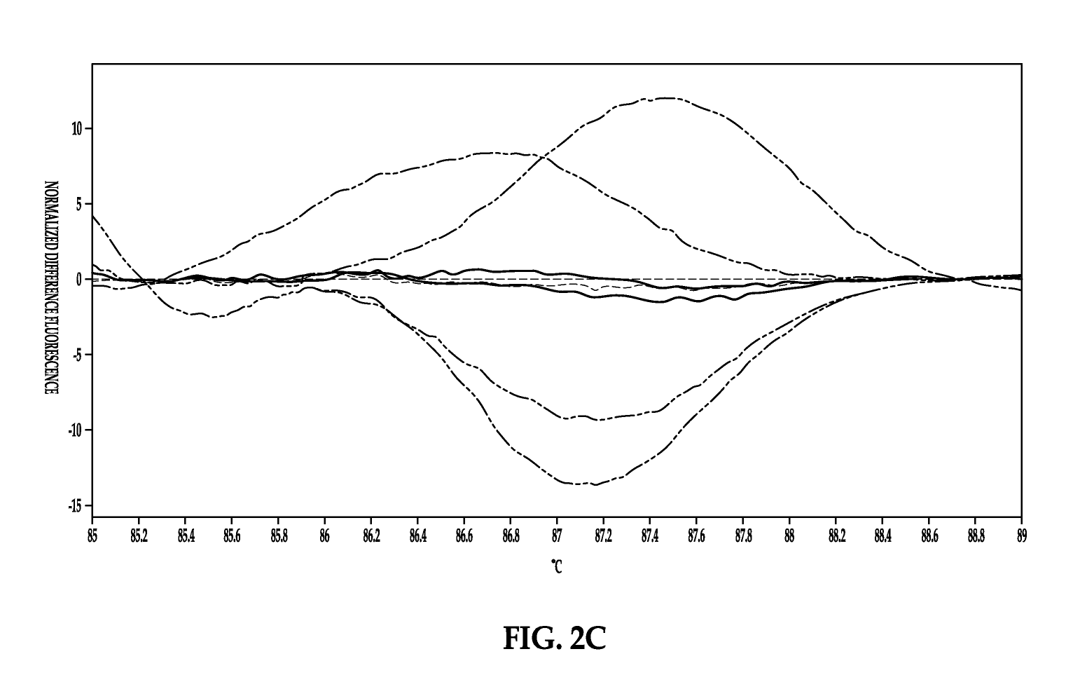

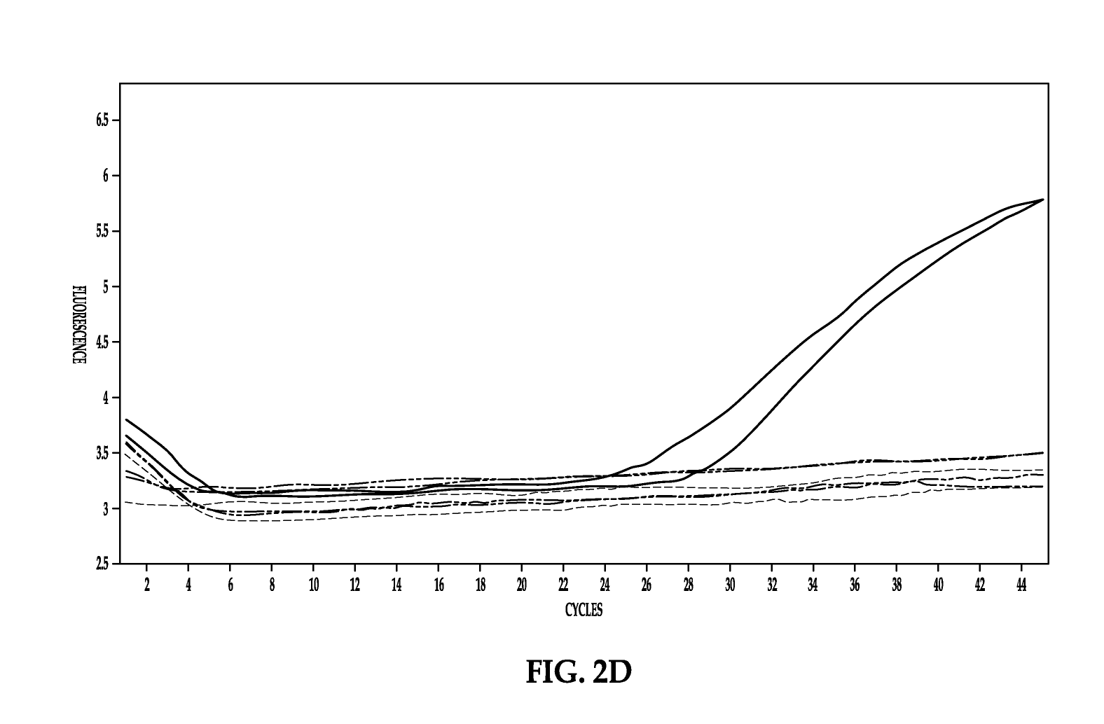

An exemplary work flow algorithm (FIG. 1) describes the assay set up and data analysis used to determine if a strain is MTBC and RIF/INH resistant. The foundation of the assay is real-time PCR, optionally SYBR based, followed by HRM analysis. As both transitions and transversion SNPs within rpoB and katG confer drug resistance, HRM analysis alone does not adequately detect all possible mutations, especially transversions, and LNA probes are optionally designed to target the most common transversion SNPs. A first tube is a multiplexed PCR reaction containing primers specific for internal fragments of IS6110 and the RRDR of rpoB from MTBC. The IS6110 marker is analyzed using melt curve analysis (MCA) of the PCR amplicons to confirm that samples are MTBC strains. MCA profiles for MTBC strains contain two peaks, one corresponding to rpoB and another to IS6110 (FIG. 2A). The primers also amplify rpoB from other NTM bacteria including Mycobacterium avium subspecies avium, Mycobacterium chelonae, and Mycobacterium abscessus (FIG. 2A) but not Mycobacterium fortuitum, Mycobacterium gordonae, or Mycobacterium intracellulare. Typically, if amplification does occur, only one curve peak is observed in the absence of IS6110 from NTM strains. When subjecting NTM strains to the assay, the rpoB amplicons have distinct melting curves as compared to MTBC, and the katG and inhA portions of the assay are not reactive to NTM strains. Due to the composition of the sample set, all 252 strains used in this study are confirmed to be MTBC.

RIF resistance is predicted based on a combination of real-time PCR, HRM analysis, and, optionally, LNA probes. HRM analysis of the rpoB marker is optionally performed to determine RIF susceptibility by observing differences in the melting profiles from 85° C. to 89° C. The HRM profile of each isolate is compared to control strains included in each run including WT, a strain containing a C>G SNP transversion mutation, and two strains that each contain an A>T SNP transversion mutation at distinct loci. Separation of melting profiles between WT strains and strains containing non-transversion SNP begins at ˜85° C. and ends at ˜89° C. The unique HRM profiles of WT strains and strains containing SNP transition and transversion mutations are shown in FIG. 2B in the “normalized graph mode”. FIG. 2C displays the same HRM profiles viewed as a “difference graph”. Both views show unique melting profiles for strains containing SNPs (-**-) when compared to WT (- - -). Those strains with melt profiles that deviate from the WT control are classified as RIF resistant by this assay and no further analysis is necessary (125 of 252 strains). However, some SNP transversions (solid) are less distinct, requiring LNA probes to detect specific SNP A>T changes at Asp516Val and His526Leu of the RRDR. A positive amplification signal in either one of the LNA probe channels (e.g. crimson channel for Asp516Val, orange channel for His526Leu) indicates RIF resistance (12 of 252 strains), whereas no amplification in either channel indicates a RIF susceptible strain (FIG. 2D).

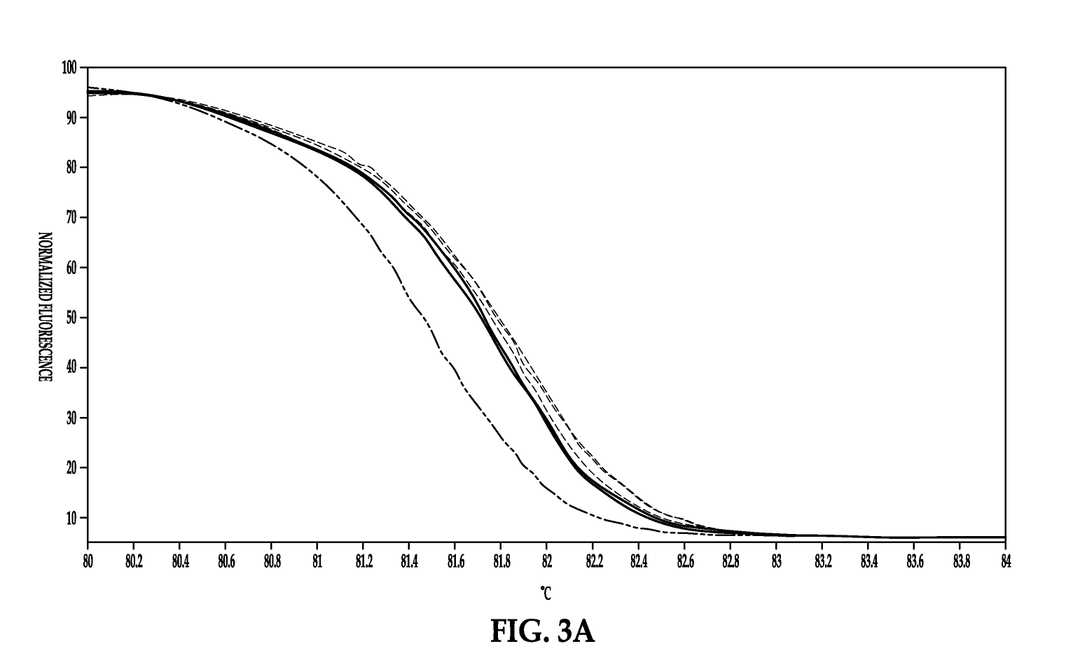

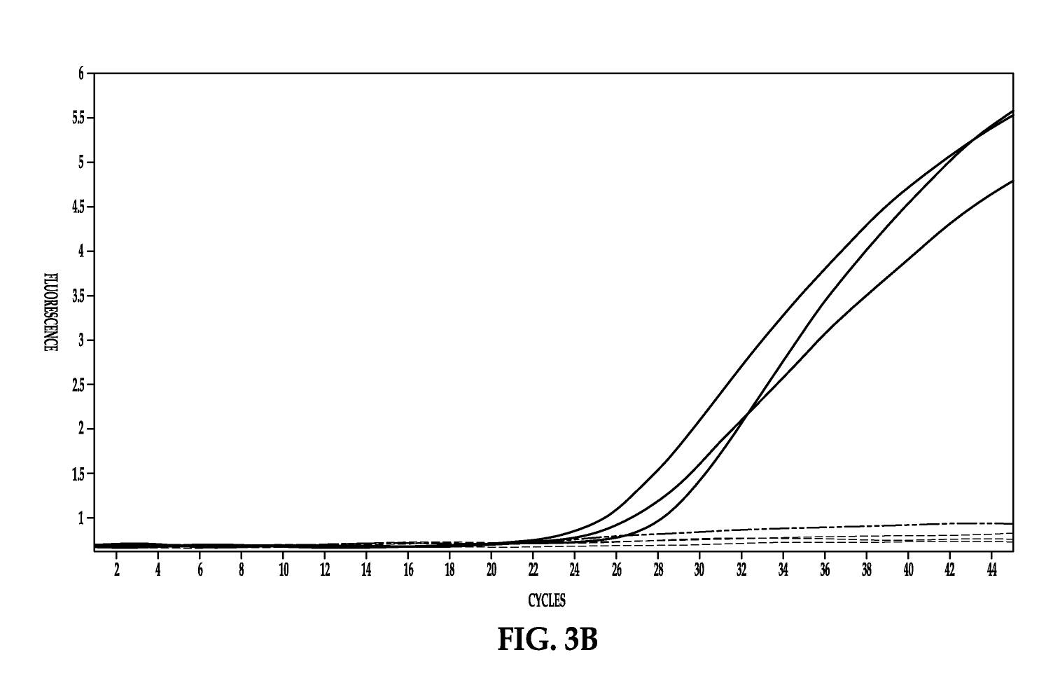

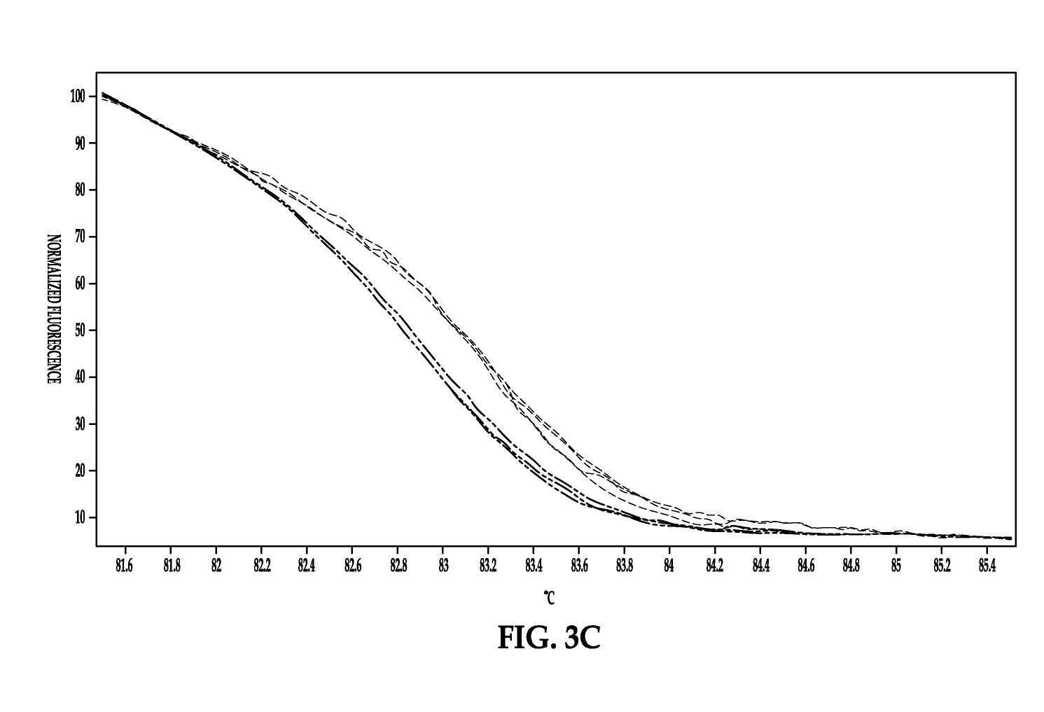

Following the classification of a strain as MTBC and the determination of RIF resistance, the real-time PCR and HRM analysis for katGlinhA is performed. The two markers are analyzed independently of each other as depicted in the exemplary algorithm shown in FIG. 1. INH resistance identified by the katG marker is detected using HRM analysis and positive amplification using an LNA probe to detect the Ser315Thr mutation. HRM analysis is done by comparing the melting pattern of each sample to those of WT, SNP transition mutation (G>A), and LNA probe SNP transversion mutation (G>C) control strains included in each run. Separation of melting profiles between WT strains (- - -) and strains containing transition SNP (-**-) mutations occur between ˜80° C. and ˜85° C., shown in FIG. 3A. Samples with melt profiles that deviate from WT are classified as resistant (2 of 252 stains). Only two strains harboring either Ser315Asn or Ser315Ile are detected by melt analysis alone. An additional strain harboring a Gly273Ser mutation is outside the target amplicon. Following HRM analysis, any samples with a melting profile indistinguishable from WT strains (solid) are further analyzed for positive amplification in the LNA probe channel. Samples with a positive signal in the LNA probe channel (red channel for Ser315Thr, solid line) are identified as INH resistant (141 of 252 strains), and those with a negative LNA probe signal are analyzed further for mutations in the inhA promoter region (FIG. 3B).

INH resistance as identified by the inhA marker is detected using HRM analysis alone. The melting profile of each sample is compared to those of WT and SNP transition (C>T or T>C) control strains included in each run. Separation of melting profiles between WT strains and strains containing mutations begins at ˜80° C. and ends at ˜86° C. FIG. 3C illustrates a “normalized graph” of the inhA HRM melting profiles between WT strains (- - -) and strains containing a mutation (-**-). Samples with melt profiles that deviate from WT are classified as resistant (29 of 252 strains). An inventive process is able to detect 10 INH resistant strains harboring mutations in only the inhA promoter region that are not associated with katG mutations. Isolates identified as containing a mutation in either katG, the inhA promoter region, or both are considered INH resistant.

Each of the 252 isolates is tested using this assay and results are compared to direct sequencing of targeted loci as well as to phenotypes determined by DST. The rpoB/1S6110 portion of the assay correctly identifies 140 of the 154 RIF resistant isolates and 96 of the 98 RIF susceptible isolates, resulting in a sensitivity of 91% and a specificity of 98%. The katGlinhA portion of the assay correctly identifies 151 of the 174 INH resistant strains and 78 of the 78 INH susceptible strains, resulting in a sensitivity of 87% and a specificity of 100%. Combined, the assay correctly identifies 126 of the 148 MDR strains and 102 of the 104 non-MDR strains, resulting in an overall sensitivity of 85% and a specificity of 98%.

### REFERENCES

1. 2003. Treatment of tuberculosis. MMWR Recomm Rep 52:1-77.
2. Bravo, L. T., M. J. Tuohy, C. Ang, R. V. Destura, M. Mendoza, G. W. Procop, S. M. Gordon, G. S. Hall, and N. K. Shrestha. 2009. Pyrosequencing for rapid detection of Mycobacterium tuberculosis resistance to rifampin, isoniazid, and fluoroquinolones. J Clin Microbiol 47:3985-90.
3. CDC. Reported Tuberculosis in the United States, 2008. Atlanta, Ga.: U.S. Department of Health and Human Services, CDC, September 2009.
4. Chung, H., Y. H. Yoon, J. J. Hwang, K. S. Cho, J. Y. Koh, and J. G. Kim. 2009. Ethambutol-induced toxicity is mediated by zinc and lysosomal membrane permeabilization in cultured retinal cells. Toxicol Appl Pharmacol 235:163-70.
5. Hoek, K. G., N. C. Gey van Pittius, H. Moolman-Smook, K. Carelse-Tofa, A. Jordaan, G. D. van der Spuy, E. Streicher, T. C. Victor, P. D. van Helden, and R. M. Warren. 2008. Fluorometric assay for testing rifampin susceptibility of Mycobacterium tuberculosis complex. J Clin Microbiol 46:1369-73.
6. Kapur, V., L. L. Li, S. Iordanescu, M. R. Hamrick, A. Wanger, B. N. Kreiswirth, and J. M. Musser. 1994. Characterization by automated DNA sequencing of mutations in the gene (rpoB) encoding the RNA polymerase beta subunit in rifampin-resistant Mycobacterium tuberculosis strains from New York City and Texas. J Clin Microbiol 32:1095-8.
7. Lacoma, A., N. Garcia-Sierra, C. Prat, J. Ruiz-Manzano, L. Haba, S. Roses, J. Maldonado, and J. Dominguez. 2008. GenoType MTBDRplus assay for molecular detection of rifampin and isoniazid resistance in Mycobacterium tuberculosis strains and clinical samples. J Clin Microbiol 46:3660-7.
8. Liew, M., R. Pryor, R. Palais, C. Meadows, M. Erali, E. Lyon, and C. Wittwer. 2004. Genotyping of single-nucleotide polymorphisms by high-resolution melting of small amplicons. Clin Chem 50:1156-64.
9. Musser, J. M. 1995. Antimicrobial agent resistance in mycobacteria: molecular genetic insights. Clin Microbiol Rev 8:496-514.
10. NCCLS. 2003. Susceptibility testing of mycobacteria, nocardiae, and other aerobic actinomycetes. Approved standard. NCCLS document M24-A. NCCLS, Wayne, Pa.
11. Ong, D. C., W. C. Yam, G. K. Siu, and A. S. Lee. Rapid Detection of Rifampicin- and Isoniazid-Resistant Mycobacterium tuberculosis by High-Resolution Melting Analysis. J Clin Microbiol.
12. Pietzka, A. T., A. Indra, A. Stoger, J. Zeinzinger, M. Konrad, P. Hasenberger, F. Allerberger, and W. Ruppitsch. 2009. Rapid identification of multidrug-resistant Mycobacterium tuberculosis isolates by rpoB gene scanning using high-resolution melting curve PCR analysis. J Antimicrob Chemother 63:1121-7.
13. Plikaytis, B. B., R. H. Gelber, and T. M. Shinnick. 1990. Rapid and sensitive detection of Mycobacterium leprae using a nested-primer gene amplification assay. J Clin Microbiol 28:1913-7.
14. Ramaswamy, S., and J. M. Musser. 1998. Molecular genetic basis of antimicrobial agent resistance in Mycobacterium tuberculosis: 1998 update. Tuber Lung Dis 79:3-29.
15. Riska, P. F., W. R. Jacobs, Jr., and D. Alland. 2000. Molecular determinants of drug resistance in tuberculosis. Int J Tuberc Lung Dis 4:S4-10.
16. Sacks, L. V., and R. E. Behrman. 2008. Developing new drugs for the treatment of drug-resistant tuberculosis: a regulatory perspective. Tuberculosis (Edinb) 88 Suppl 1:S93-100.
17. Vignal, A., D. Milan, M. SanCristobal, and A. Eggen. 2002. A review on SNP and other types of molecular markers and their use in animal genetics. Genet Sel Evol 34:275-305.
18. Vilcheze, C, and W. R. Jacobs, Jr. 2007. The mechanism of isoniazid killing: clarity through the scope of genetics. Annu Rev Microbiol 61:35-50.
19. WHO. 2008. Anti-tuberculosis drug resistance in the world: fourth global report.
20. WHO. 2009. Global Tuberculosis Control: epidemiology, strategy, financing. WHO Report 2009.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified.

Patents and publications mentioned in the specification are indicative of the levels of those skilled in the art to which the invention pertains. These patents and publications are incorporated herein by reference to the same extent as if each individual application or publication was specifically and individually incorporated herein by reference for the entirety of their teaching.

The foregoing description is illustrative of particular embodiments of the invention, but is not meant to be a limitation upon the practice thereof. The following claims, including all equivalents thereof, are intended to define the scope of the invention.

1. A process for detecting drug resistance in a Mycobacterium tuberculosis strain, comprising:
exposing a DNA sample to a primer pair comprising a forward primer and a reverse primer under conditions conducive to a polymerase chain reaction to yield an amplicon, wherein said primer pair is specific to the region of rpoB, katG or inhA promoter of the Mycobacterium tuberculosis strain; and
detecting said amplicon indicative of the Mycobacterium tuberculosis strain.

2. The process of claim 1 wherein said primer pair is: SEQ ID NO: 7 and SEQ ID NO: 8; SEQ ID NO: 13 and SEQ ID NO: 14, or SEQ ID NO: 16 and SEQ ID NO: 17.

3. The process of claim 1 further comprising exposing the sample to an IS6110 insertion element forward primer and an IS6110 insertion element reverse primer under the conditions conducive to polymerase chain reaction to yield an amplicon and to determine a strain as a MTBC or NTM strain of mycobacteria.

4. The process of claim 3 wherein said IS6110 insertion element forward primer has the sequence of SEQ ID NO: 9.

5. The process of claim 3 wherein said IS6110 insertion element reverse primer has the sequence of SEQ ID NO: 10.

6. The process of claim 1 further comprising performing high resolution melt analysis on a double-stranded product comprising said amplicon.

7. The process of claim 6 wherein said high resolution melt analysis is performed between 80 degrees Celsius and 89 degrees Celsius.

8. The process of claim 6 wherein said high resolution melt analysis is performed at a rate of 0.02 degrees Celsius per step.

9. The process of claim 1 further comprising exposing said amplicon to a probe targeting a specific locus in one of rpoB, katG or inhA promoter.

10. The process of claim 9 wherein said probe includes a locked nucleic acid.

11. The process of claim 9 wherein said katG probe has the sequence of SEQ ID NO: 15.

12. The process of claim 9 wherein said rpoB probe has the sequence of SEQ ID NO: 11 or SEQ ID NO: 12.

13. The process of claim 1 further comprising subjecting said DNA sample to a DNA sequencing reaction.

14. The process of claim 13 wherein said DNA sequencing reaction uses a primer pair selected from the sequences of SEQ ID NO: 1 and SEQ ID NO: 2; SEQ ID NO: 3 and SEQ ID NO: 4, or SEQ ID NO: 5 and SEQ ID NO: 6; or combinations of said pairs.

15. The process of claim 1 wherein said DNA sample is obtained by selective amplification of a region of a Mycobacterium tuberculosis strain genome.

16. The process of claim 15 wherein said selective amplification comprises:
exposing said genome to a primer pair comprising a forward primer and a reverse primer under conditions conducive to a polymerase chain reaction to yield said DNA sample, wherein said primer pair is specific to the region of rpoB, katG or inhA promoter of the Mycobacterium tuberculosis strain.

17. The process of claim 16 wherein said primer pair comprises nucleotide sequences having the sequence of SEQ ID NO: 1 and SEQ ID NO: 2; SEQ ID NO: 3 and SEQ ID NO: 4, or SEQ ID NO: 5 and SEQ ID NO: 6.

18.-21. (canceled)

22. A kit for detecting a Mycobacterium tuberculosis strain comprising:
a forward primer—reverse primer pair of SEQ ID NO: 7—SEQ ID NO: 8, SEQ ID NO: 13—SEQ ID NO: 14, or SEQ ID NO: 16—SEQ ID NO: 17; and
a detectable probe specific for specific SNPs in a Mycobacterium tuberculosis strain or generic to more than one Mycobacterium tuberculosis strain.

23. The kit of claim 22 wherein said probe has the sequence of SEQ ID NO: 11, SEQ ID NO: 12, or SEQ ID NO: 15.

24. The kit of claim 22 further comprising a primer having a sequence of SEQ ID NO: 1, SEQ ID NO: 2, SEQ ID NO: 3, SEQ ID NO: 4, SEQ ID NO: 5, SEQ ID NO: 6, or combinations of primers.

25.-49. (canceled)
